(19) United States
(12) Patent Application Publication
(10) Pub. No.: US 2026/0367263 A1
(43) Pub. Date: Nov. 19, 2026

(54) SENSING MATERIALS FOR GAS AND RADIATION DETECTION AND SENSING DEVICES FOR GAS AND RADIATION DETECTION COMPRISING THE SAME

(71) Applicant: University-Industry Cooperation Group of Kyung Hee University, Gyeonggi-do (KR)

(72) Inventors: Jung Ho YUN, Gyeonggi-do (KR); Su Min LEE, Gyeonggi-do (KR)

(21) Appl. No.: 19/676,209

(22) Filed: May 13, 2026

(30) Foreign Application Priority Data
May 15, 2025 (KR) 10-2025-0063212
May 12, 2026 (KR) 10-2026-0085050

(51) Int. Cl. G01T 1/24; G01N 33/00

(52) U.S. Cl. CPC G01T 1/244; G01N 33/0062

(57) ABSTRACT

The present disclosure provides a sensor material capable of complexly and easily detecting gas and radiation, and a sensor including the same.

### CROSS-REFERENCE TO RELATED APPLICATIONS

This application claims priority from and the benefit of Korean Patent Application No. 10-2025-0063212, filed on May 25, 2025, and Korean Patent Application No. 10-2026-0085050, filed on May 15, 2026, which is hereby incorporated by reference for all purposes as if set forth herein.

### BACKGROUND

### 1. Technical Field

The present disclosure relates to a sensor material for detecting gas and radiation, and a sensor including the same.

### 2. Related Art

Recently, rapid detection of complex hazardous substances is being utilized as a key means for minimizing human casualties in various national disaster situations such as chemical plant explosion and leakage accidents, large-scale forest fires, and fires in subways and tunnels, as well as in military operational environments responding to chemical, biological, radiological, and nuclear (CBRN) threats, and in extreme battlefield environments such as chemical warfare and radiological warfare. Accordingly, remote hazardous gas detection equipment is installed and used around nuclear power plants or near national industrial complexes in order to detect hazardous gases in the atmosphere.

In particular, when a hazardous gas leakage accident occurs, the detection equipment is utilized to mainly analyze the amount of hazardous gas and the direction in which the hazardous gas diffuses, thereby evacuating residents located at a short distance from the hazardous gas leakage accident site and securing on-site safety.

However, conventional remote hazardous gas detection equipment detects only hazardous gases in the atmosphere and does not have a radiation detection function. Thus, when radiation leakage occurs, there is no function for detecting the radiation leakage, resulting in a problem in that separate radiation detection equipment must be additionally provided and installed.

This is a structural limitation that identically appears not only around nuclear power plants but also in complex threat situations such as radioactive material transport accidents, areas near nuclear facilities, and radiological terrorism, and there is a problem in that complex risk factors cannot be simultaneously monitored with a single device.

Accordingly, there is a need to develop a sensor capable of complexly detecting gas and radiation.

### SUMMARY

The present disclosure is to provide a sensor material capable of complexly and easily detecting gas and radiation, and a sensor including the same.

However, the problems to be solved by the present disclosure are not limited to the above-mentioned problem, and other problems not mentioned will be clearly understood by those skilled in the art from the following description.

An embodiment of the present disclosure provides a material for a gas and radiation detection sensor comprising: a first electrode; a second electrode; a perovskite sensing layer including perovskite and configured to sense gas and radiation; and a functional layer provided on one surface of the perovskite sensing layer and including a functional material.

According to an embodiment of the present disclosure, the perovskite sensing layer may comprise at least one of perovskites represented by Chemical Formula 1 to Chemical Formula 4 below:

ABX₃ [Chemical Formula 1]

A′₂Aₙ₋₁BₙX₃ₙ₊₁ [Chemical Formula 2-1]

A″Aₙ₋₁BₙX₃ₙ₊₁ [Chemical Formula 2-2]

A₄BX₆ [Chemical Formula 3-1]

AB₂X₅ [Chemical Formula 3-2]

A₃B₂X₉ [Chemical Formula 4]
wherein A, A′, and A″ each independently comprise at least one of an organic ammonium ion and an alkali metal, B comprises at least one metal of Pb, Sn, Bi, and Sb, X is a halogen anion, and n is 0 or an integer of 1 to 10.

According to an embodiment of the present disclosure, the functional layer may be provided on a part of one surface of the perovskite sensing layer.

According to an embodiment of the present disclosure, the functional material may comprise an ammonium halide-based compound.

According to an embodiment of the present disclosure, the functional material may comprise a gas sensing-enhancing material including at least one of a thiol group and a carboxyl group.

According to an embodiment of the present disclosure, the functional material may comprise at least one of an electron transport-enhancing material and a hole transport-enhancing material.

According to an embodiment of the present disclosure, the functional material may comprise a radiation-resistant material including a lanthanide metal ion.

According to an embodiment of the present disclosure, the functional layer may have a thickness of 1 nm or more and 50 nm or less.

According to an embodiment of the present disclosure, the first electrode and the second electrode may be provided to face each other, and the perovskite sensing layer and the functional layer may be provided between the first electrode and the second electrode.

According to an embodiment of the present disclosure, the first electrode and the second electrode may be provided to face each other, the first electrode may be provided on one surface of the functional layer provided on a first region of the perovskite sensing layer, and the second electrode may be provided on a second region of the perovskite sensing layer.

An embodiment of the present disclosure provides a gas and radiation detection sensor comprising the material for a gas and radiation detection sensor.

The sensor material and the sensor including the same according to an embodiment of the present disclosure have an advantage of being able to easily detect gas and radiation.

The effects of the present disclosure are not limited to the effects mentioned above, and effects not mentioned will be clearly understood by those skilled in the art from the present specification and the accompanying drawings.

### BRIEF DESCRIPTION OF DRAWINGS

FIG. 1 is a diagram schematically illustrating a cross-sectional view of a material for a gas and radiation detection sensor according to an embodiment of the present disclosure.

FIG. 2 is a diagram schematically illustrating a cross-sectional view of a material for a gas and radiation detection sensor according to another embodiment of the present disclosure.

FIG. 3 is a diagram schematically illustrating a perspective view of a material for a gas and radiation detection sensor according to another embodiment of the present disclosure.

FIG. 4 is a diagram illustrating results of a gas detection performance evaluation of a material for a gas and radiation detection sensor manufactured in an Example of the present disclosure.

FIG. 5 is a diagram illustrating results of a gas detection performance evaluation of a material for a gas and radiation detection sensor manufactured in a Comparative Example.

FIG. 6 is a diagram comparing results of a gas detection performance evaluation of a material for a gas and radiation detection sensor manufactured in an Example of the present disclosure and a material for a gas and radiation detection sensor manufactured in a Comparative Example.

FIG. 7 is a diagram illustrating results of a radiation detection performance evaluation of a material for a gas and radiation detection sensor manufactured in an Example of the present disclosure.

### DETAILED DESCRIPTION

Throughout the present specification, when a part is described as “including” a certain component, it means that other components may be further included, rather than excluded, unless otherwise stated.

Throughout the present specification, when a member is described as being located “on” another member, it includes not only a case where the member is in contact with the other member, but also a case where a third member exists between the two members.

Throughout the present specification, the term “parts by weight” may refer to a ratio of weights among components.

Throughout the present specification, terms including ordinal numbers, such as “first” and “second”, are used only for the purpose of distinguishing one component from another component, and are not limited by the ordinal numbers. For example, within the scope of the disclosure, a first component may be referred to as a second component, and similarly, a second component may be referred to as a first component.

Hereinafter, the present disclosure will be described in more detail.

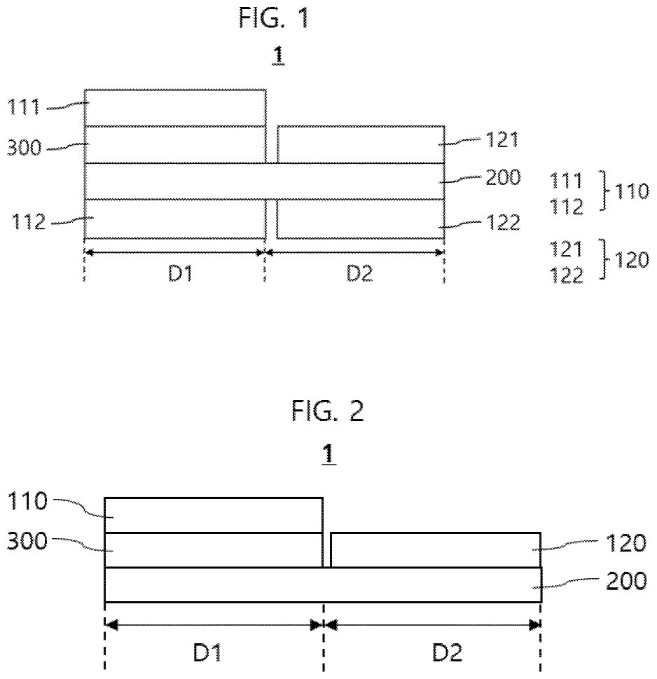

FIG. 1 is a diagram schematically illustrating a cross-sectional view of a material for a gas and radiation detection sensor according to an embodiment of the present disclosure, and FIG. 2 is a diagram schematically illustrating a cross-sectional view of a material for a gas and radiation detection sensor according to another embodiment of the present disclosure. Referring to FIGS. 1 and 2, positions at which a first electrode 110 and a second electrode 120 are provided in a material 1 for a gas and radiation detection sensor may be different, and a detailed description thereof will be provided later.

An embodiment of the present disclosure provides a material for a gas and radiation detection sensor including: a first electrode; a second electrode; a perovskite sensing layer including perovskite and configured to sense gas and radiation; and a functional layer provided on one surface of the perovskite sensing layer and including a functional material.

The material for a sensor according to an embodiment of the present disclosure has an advantage of being able to easily detect gas and radiation.

According to an embodiment of the present disclosure, the material for a gas and radiation detection sensor includes the perovskite sensing layer, thereby being capable of complexly sensing gas and radiation. In this case, the material for a gas and radiation detection sensor includes a functional layer provided on one surface of the perovskite sensing layer, thereby additionally implementing various functions in the material for a sensor. Specifically, the functional layer may control a surface defect state of the perovskite sensing layer. Thereby, sensing capability of the material for a sensor can be further improved. In addition, the functional layer may enhance an interaction between the perovskite sensing layer and a gas, thereby improving a sensing function. In addition, the functional layer may improve the electron transport and/or hole transport, thereby increasing sensing efficiency. In addition, the functional layer may impart a radiation-resistant effect on the perovskite sensing layer.

Referring to FIGS. 1 and 2, the material 1 for a gas and radiation detection sensor may include a perovskite sensing layer 200.

According to an embodiment of the present disclosure, the perovskite sensing layer may include at least one of perovskites represented by Chemical Formula 1 to Chemical Formula 4 below:

ABX₃ [Chemical Formula 1]

A′₂Aₙ₋₁BₙX₃ₙ₊₁ [Chemical Formula 2-1]

A″Aₙ₋₁BₙX₃ₙ₊₁ [Chemical Formula 2-2]

A₄BX₆ [Chemical Formula 3-1]

AB₂X₅ [Chemical Formula 3-2]

A₃B₂X₉ [Chemical Formula 4]
wherein A, A′, and A″ each independently include at least one of an organic ammonium ion and an alkali metal, B includes at least one metal of Pb, Sn, Bi, and Sb, X is a halogen anion, and n is 0 or an integer of 1 to 10.

Specifically, in the above Chemical Formulas, A, A′, and A″ may each independently include an organic ammonium ion, wherein the organic ammonium ion may include at least one of a methylammonium ion, an ethylammonium ion, a propylammonium ion, a phenylammonium ion, and a formamidinium ion. The alkali metal may include at least one of Cs, Rb, Na, K, and Li.

According to an embodiment of the present disclosure, the perovskite sensing layer may be manufactured using a precursor material capable of forming perovskite. In addition, the perovskite sensing layer may be manufactured utilizing a perovskite single crystal. For example, the sensing layer may be formed by growing perovskite in a single crystal form. In this case, the perovskite may be grown in a crystal form from a perovskite precursor solution using an inverse temperature crystallization, a slow cooling crystallization, a solvent evaporation crystallization, or an anti-solvent assisted method. Alternatively, the sensing layer may be formed by melting a perovskite single crystal and using a solution process. In addition, a perovskite sensing layer in a thin film form may be formed from a perovskite precursor solution using a solution process.

The perovskite sensing layer including a perovskite single crystal has low defects and can implement an appropriate thickness, and thus can more effectively detect radiation as compared to a thin film-type sensing layer including a perovskite polycrystal.

According to an embodiment of the present disclosure, the sensing layer may be formed using a perovskite precursor solution. The perovskite precursor solution may include a precursor material for forming the compounds represented by Chemical Formulas 1 to 4 and a solvent. As the precursor material, any material used in the art to manufacture the compounds represented by Chemical Formulas 1 to 4 may be used without limitation. For example, the precursor material may include methylammonium bromide (MABr) and lead bromide (PbBr₂).

In addition, the solvent may be one used in the art, and for example, the solvent may include at least one of dimethylformamide (DMF), dimethyl sulfoxide (DMSO), γ-butyrolactone (GBL), N,N-dimethylacetamide, N-methyl-2-pyrrolidone (NMP), 1,3-dimethyl-2-imidazolidinone (DMI), dimethylacetamide (DMA), 2-mercaptoethanol (2ME), hexamethylphosphoramide (HMPA), acetonitrile, and methylamine. However, the type of the solvent is not limited to those described above.

According to an embodiment of the present disclosure, a content of the perovskite precursor material included in the perovskite precursor solution may be 0.5 M or more and 3.0 M or less, 0.8 M or more and 2.5 M or less, 1.0 M or more and 2.0 M or less, 1.2 M or more and 1.5 M or less, 0.5 M or more and 2.0 M or less, 0.7 M or more and 2.7 M or less, 1.0 M or more and 2.3 M or less, 1.0 M or more and 3.0 M or less, or 1.5 M or more and 2.5 M or less. When the content of the precursor material included in the perovskite precursor solution is within the above-described range, processability of manufacturing the sensing layer can be improved, and the perovskite sensing layer can be stably formed from the perovskite precursor solution.

According to an embodiment of the present disclosure, the perovskite sensing layer may be manufactured by generating perovskite crystals from the perovskite precursor solution using an inverse temperature crystallization method. Specifically, the perovskite sensing layer may be formed at a temperature of 20 °C or more and 100 °C or less, 20 °C or more and 80 °C or less, or 20 °C or more and 60 °C or less. In this case, a heating rate for the perovskite precursor solution may be 0.5 °C/min or more and 5 °C/min or less, 0.8 °C/min or more and 4 °C/min or less, 1 °C/min or more and 3 °C/min or less, 0.5 °C/min or more and 3 °C/min or less, 0.8 °C/min or more and 2 °C/min or less, or 3 °C/min or more and 5 °C/min or less. When conditions for generating the crystals are controlled within the above-described ranges, the perovskite sensing layer can be stably formed.

According to an embodiment of the present disclosure, the perovskite sensing layer may have a thickness of 10 nm or more and 1 cm or less. Specifically, when the perovskite sensing layer includes perovskite in a single crystal form, the perovskite sensing layer may have a thickness of 1 μm or more and 1 cm or less. Meanwhile, when the perovskite sensing layer is provided in a thin film form including polycrystalline perovskite, the perovskite sensing layer may have a thickness of 100 nm or more and 1,000 nm or less. When the thickness of the perovskite sensing layer is within the above-described range, the material for a sensor can effectively detect gas and radiation.

According to an embodiment of the present disclosure, the functional layer may include, as a functional material, at least one of an ammonium halide-based compound, a gas sensing-enhancing material, an electron transport-enhancing material, a hole transport-enhancing material, and a radiation-resistant material. By selecting and using the type of the functional material included in the functional layer from those described above, a function suitable for the purpose of using the material for a sensor can be effectively implemented.

According to an embodiment of the present disclosure, the functional layer may be formed on the perovskite sensing layer through a deposition method used in the art. For example, the functional layer may be manufactured by coating the functional material on the perovskite sensing layer using a solution process, sputtering, an e-beam deposition method, or a vacuum thermal deposition method. Here, the solution process may include at least one of spin coating, dip coating, spray coating, doctor blade coating, roll coating, bar coating, gravure coating, and slot-die coating.

According to an embodiment of the present disclosure, the functional layer may be provided on a part of one surface of the perovskite sensing layer.

Referring to FIGS. 1 and 2, the perovskite sensing layer 200 may be divided into a first region D1 and a second region D2. In this case, a functional layer 300 may be provided on the first region D1 of the perovskite sensing layer 200. The first region D1 of the perovskite sensing layer 200 may correspond to a region for detecting gas, and the second region D2 of the perovskite sensing layer 200 may correspond to a region for detecting radiation. That is, the material 1 for a gas and radiation detection sensor may detect gas in the first region D1 and detect radiation in the second region D2, thereby being capable of complexly and simultaneously detecting gas and radiation.

According to an embodiment of the present disclosure, the functional material may include an ammonium halide-based compound. Specifically, the functional layer may include an ammonium halide-based compound as a surface defect control material for controlling a surface defect state of the perovskite sensing layer. As described above, by the functional layer including the ammonium halide-based compound, the surface defect state of the perovskite sensing layer can be controlled, thereby further improving sensing capability of the material for a sensor.

According to an embodiment of the present disclosure, the ammonium halide-based compound may include a compound represented by Chemical Formula 5 below:

R₂—R₁—NH₃⁺X⁻ [Chemical Formula 5]
wherein R₁ is a linear or branched alkylene having 1 to 5 carbon atoms, and R₂ is a substituted or unsubstituted aromatic ring having 6 to 30 carbon atoms, or a substituted or unsubstituted cyclic alkyl having 5 to 10 carbon atoms. Here, the substituted aromatic ring may be substituted with at least one halogen ion. In addition, the substituted cyclic alkyl may be substituted with at least one halogen ion. The aromatic ring may be a benzene ring, and the cyclic alkyl may be cyclohexyl. In addition, in Chemical Formula 5 above, X is a halogen element and may be I, Br, or Cl.

Specifically, the halogenated compound may include at least one of phenethylammonium iodide (PEAI), phenethylammonium bromide (PEABr), pentafluorophenethylammonium iodide (F-PEAI), pentafluorophenethylammonium bromide (F-PEABr), butylammonium iodide (BAI), butylammonium bromide (BABr), octylammonium iodide (OAI), and phenylenediammonium iodide (PDAI).

According to an embodiment of the present disclosure, the functional material may include a gas sensing-enhancing material including at least one of a thiol group and a carboxyl group. As described above, by the functional layer including the gas sensing-enhancing material, an interaction between the perovskite sensing layer and a gas can be enhanced. Thereby, a sensing function of the material for a sensor can be further improved.

Specifically, the gas sensing-enhancing material including a thiol group may include at least one of 2-mercaptoethanol, 3-mercaptopropionic acid, cysteine, glutathione, and thiourea. In addition, the gas sensing-enhancing material including a carboxyl group may include at least one of benzoic acid, succinic acid, and citric acid.

According to an embodiment of the present disclosure, the functional material may include at least one of an electron transport-enhancing material and a hole transport-enhancing material. As described above, when the functional layer includes at least one of an electron transport-enhancing material and a hole transport-enhancing material, the electron transport and/or hole transport can be improved, thereby increasing sensing efficiency.

According to an embodiment of the present disclosure, the electron transport-enhancing material may include at least one of a fullerene derivative selected from 6,6-phenyl-C61-butyric acid methyl ester (PCBM(C61)), PCBM(C60), PCBM(C70), PCBM(C71), PCBM(C76), PCBM(C80), PCBM(C82), indene-C60 bisadduct (ICBA), and 6,6-phenyl-C61-butyric acid cholesteryl ester (PCBCR), polybenzimidazole, perylene, poly[[N,N′-bis(2-octyldodecyl)-naphthalene-1,4,5,8-bis(dicarboximide)-2,6-diyl]-alt-5,5′-(2,2′-bithiophene)] (NDI2OD-T2), naphthalene diimide (NDI)-selenophene copolymer (PNDIS-HD), poly[(E)-2,7-bis(2-decyltetradecyl)-4-methyl-9-(5-(2-(5-methylthiophen-2-yl)vinyl)thiophen-2-yl)benzo[lmn][3,8]phenanthroline-1,3,6,8(2H,7H)-tetraone] (PNDI-TVT), poly[[N,N′-bis(2-hexyldecyl)naphthalene-1,4,5,8-bis(dicarboximide)-2,6-diyl]-alt-5,5′-thiophene] (PNDI2HD-T), NiOx, BCP (Bathocuproine), and a p-type oxide semiconductor.

According to an embodiment of the present disclosure, the hole transport-enhancing material may include at least one of MoO₃, V₂O₅, MEO-2PAC, spiro-OMeTAD (2,2′,7,7′-tetrakis(N,N-p-dimethoxyphenylamino)-9,9′-spirobifluorene), P3HT (poly[3-hexylthiophene]), PCPDTBT (poly[2,6-(4,4-bis-(2-ethylhexyl)-4H-cyclopenta [2,1-b; 3,4-b′]dithiophene)-alt-4,7(2,1,3-benzothiadiazole)]), PCDTBT (poly[N-9′-heptadecanyl-2,7-carbazole-alt-5,5-(4′,7′-di-2-thienyl-2′,1′,3′-benzothiadiazole)]), PTAA (poly(triarylamine)), spiro-OMeTAL, poly[bis(4-phenyl)(2,4,6-trimethylphenyl)amine]), PEDOT:PSS(Poly(3,4-ethylenedioxythiophene doped with poly(styrenesulfonic acid), cyclopenta-[2,1-b;3,4-b′]-dithiophene, benzothiadiazole, diketopyrrolopyrrole, thieno[3,4-b]thiophene, benzodithiophene, carbazole, fluorene, PEDOT:PSS (poly(3,4-ethylenedioxythiophene) poly(styrenesulfonate), PS-PAA (polystyrene polyacrylic acid block copolymer), poly-TPD (poly(4-butylphenyl-diphenyl-amine)), PPV (polyphenylene vinylene), PVP (polyvinylpyrrolidone), NiOx, and a p-type oxide semiconductor.

According to an embodiment of the present disclosure, the functional material may include a radiation-resistant material including a lanthanide metal ion. As described above, when the functional layer includes the radiation-resistant material, a radiation-resistant effect on the perovskite sensing layer can be effectively imparted. Specifically, the radiation-resistant material may include at least one of a cerium ion (Ce³⁺), a europium ion (Eu³⁺), a neodymium ion (Nd³⁺), a samarium ion (Sm³⁺), and a ytterbium ion (Yb³⁺).

According to an embodiment of the present disclosure, the functional layer may have a thickness of 1 nm or more and 100 nm or less. Specifically, the functional layer may have a thickness of 1 nm or more and 80 nm or less, 1 nm or more and 60 nm or less, 1 nm or more and 40 nm or less, 1 nm or more and 20 nm or less, 1 nm or more and 10 nm or less, 5 nm or more and 50 nm or less, 5 nm or more and 35 nm or less, or 5 nm or more and 15 nm or less. By controlling the thickness of the functional layer within the above-described ranges, the overall thickness and weight of the material for a sensor can be effectively reduced. In addition, when the thickness of the functional layer is within the above-described ranges, a desired function can be effectively imparted to the material for a sensor.

According to an embodiment of the present disclosure, a ratio of a thickness of the functional layer to a thickness of the perovskite sensing layer may be 1:100,000 to 1:2,000,000, 1:500,000 to 1:1,800,000, 1:700,000 to 1:1,500,000, 1:100,000 to 1:1,200,000, or 1:1,000,000 to 1:2,000,000. When the ratio of the thickness of the perovskite sensing layer to the thickness of the functional layer is within the above-described range, an effect intended to be implemented through the functionality can be maximized, and the overall thickness and weight of the material for a gas and radiation detection sensor can be effectively reduced. In addition, the material for a sensor can more effectively detect gas and radiation.

According to an embodiment of the present disclosure, the material for a gas and radiation detection sensor may include a first electrode and a second electrode. The first electrode and the second electrode may be manufactured by a method used in the art. For example, the first electrode and the second electrode may be manufactured by a vacuum thermal deposition method.

According to an embodiment of the present disclosure, the first electrode may include, as a material for forming the first electrode, at least one of indium tin oxide (ITO), fluorine-doped tin oxide (FTO), antimony tin oxide (ATO), gallium tin oxide (GTO), zinc tin oxide (ZTO), gallium-doped ZTO (ZTO: Ga), indium zinc oxide (IZO), aluminum zinc oxide (AZO), boron-doped zinc oxide (BZO), niobium titanium oxide (NTO), indium gallium zinc oxide (IGZO), and indium zinc tin oxide (IZTO).

According to an embodiment of the present disclosure, the second electrode may include, as a material for forming the second electrode, at least one of silver (Ag), aluminum (Al), platinum (Pt), tungsten (W), copper (Cu), molybdenum (Mo), gold (Au), nickel (Ni), palladium (Pd), indium (In), ruthenium (Ru), lead (Pb), rhodium (Rh), iridium (Ir), osmium (Os), and carbon (C). In addition, the transparent electrode may include, as a material for forming the second electrode, at least one of indium tin oxide (ITO), fluorine-doped tin oxide (FTO), antimony tin oxide (ATO), gallium tin oxide (GTO), zinc tin oxide (ZTO), gallium-doped ZTO (ZTO: Ga), indium zinc oxide (IZO), aluminum zinc oxide (AZO), boron-doped zinc oxide (BZO), niobium titanium oxide (NTO), indium gallium zinc oxide (IGZO), and indium zinc tin oxide (IZTO).

According to an embodiment of the present disclosure, each of the first electrode and the second electrode may independently have a thickness of 30 nm or more and 150 nm or less, 50 nm or more and 130 nm or less, 70 nm or more and 110 nm or less, 30 nm or more and 100 nm or less, 45 nm or more and 85 nm or less, or 100 nm or more and 150 nm or less. When the thicknesses of the first electrode and the second electrode are within the above-described ranges, the overall thickness of the material for a gas and radiation detection sensor can be effectively reduced.

FIG. 1 shows an example of a vertical structure of the material for a gas and radiation detection sensor according to an embodiment of the present disclosure.

According to an embodiment of the present disclosure, the first electrode may include a 1-1 unit electrode and a 1-2 unit electrode, and the second electrode may include a 2-1 unit electrode and a 2-2 unit electrode, wherein the perovskite sensing layer and the functional layer may be provided between the 1-1 unit electrode and the 1-2 unit electrode, and the perovskite sensing layer may be provided between the 2-1 unit electrode and the 2-2 unit electrode.

Referring to FIG. 1, the functional layer 300 may be provided on a first region D1 of the perovskite sensing layer 200. Meanwhile, a region in which the functional layer 300 is not provided may be defined as a second region D2. The first electrode 110 may include a 1-1 unit electrode 111 and a 1-2 unit electrode 112 provided as a pair. In this case, the perovskite sensing layer 200 and the functional layer 300 may be provided between the 1-1 unit electrode 111 and the 1-2 unit electrode 112. In addition, the second electrode 120 may include a 2-1 unit electrode 121 and a 2-2 unit electrode 122 provided as a pair. The perovskite sensing layer 200 may be provided between the 2-1 unit electrode 121 and the 2-2 unit electrode 122. That is, the first electrode 110 may be provided on the first region D1, and the second electrode 120 may be provided on the second region D2.

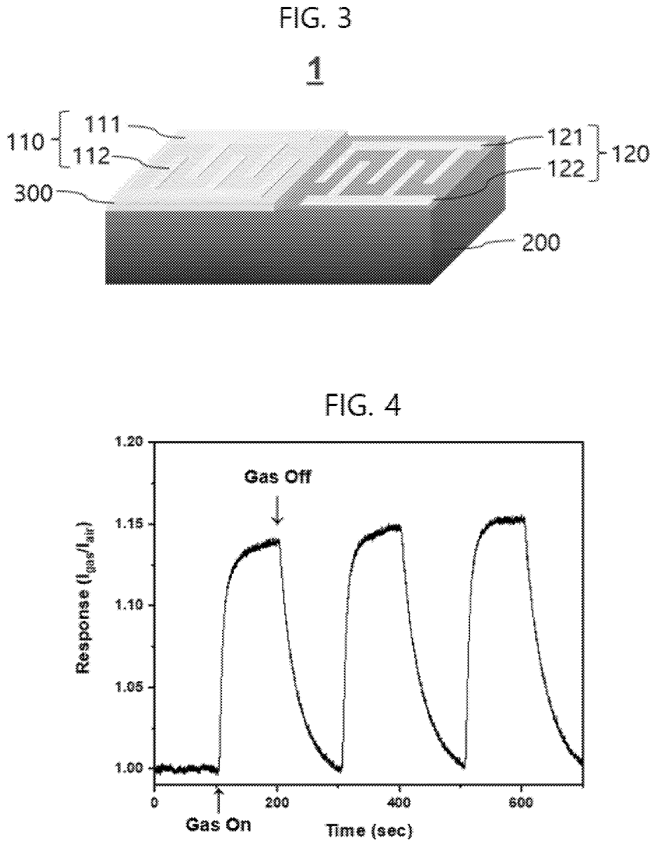

FIGS. 2 and 3 show an example of an interdigitated structure of the material for a gas and radiation detection sensor according to an embodiment of the present disclosure.

According to an embodiment of the present disclosure, the first electrode and the second electrode may be provided to face each other, the first electrode may be provided on one surface of the functional layer provided on the first region of the perovskite sensing layer, and the second electrode may be provided on the second region of the perovskite sensing layer.

FIG. 3 is a diagram schematically illustrating a perspective view of a material for a gas and radiation detection sensor according to another embodiment of the present disclosure.

Referring to FIGS. 2 and 3, the first electrode 110 and the second electrode 120 may be provided to face each other, the first electrode 110 may be provided on one surface of the functional layer provided on the first region D1 of the perovskite sensing layer 200, and the second electrode 120 may be provided on the second region D2 of the perovskite sensing layer 200.

Depending on installation conditions of the material for a gas and radiation detection sensor, a form in which the first electrode and the second electrode are provided may be set as shown in FIGS. 1 and 2.

According to an embodiment of the present disclosure, the first electrode may include a pair of first unit electrodes, and the second electrode may include a pair of second unit electrodes. Referring to FIG. 3, the first electrode 110 may include a 1-1 unit electrode 111 and a 1-2 unit electrode 112 provided as a pair. The second electrode 120 may include a 2-1 unit electrode 121 and a 2-2 unit electrode 122 provided as a pair.

According to an embodiment of the present disclosure, the material for a gas and radiation detection sensor may further include a substrate. The substrate may be provided on the other surface of the perovskite sensing layer. That is, the perovskite sensing layer may be provided between the functional layer and the substrate. As the substrate, any substrate used for a material for a sensor in the art may be used without limitation. For example, the substrate may include, as a material for forming the substrate, at least one of glass, polyethylene terephthalate (PET), polyethylene sulfone (PES), polyethylene naphthalate (PEN), polycarbonate (PC), polymethyl methacrylate (PMMA), polyimide (PI), ethylene vinyl acetate (EVA), amorphous polyethylene terephthalate (APET), polypropylene terephthalate (PPT), polyethylene terephthalate glycerol (PETG), polycyclohexylene dimethylene terephthalate (PCTG), modified triacetyl cellulose (TAC), cycloolefin polymer (COP), cycloolefin copolymer (COC), dicyclopentadiene polymer (DCPD), cyclopentadiene polymer (CPD), polyarylate (PAR), polyetherimide (PEI), polydimethylsiloxane (PDMS), silicone resin, fluororesin, and modified epoxy resin.

According to an embodiment of the present disclosure, the material for a gas and radiation detection sensor may include, in addition to the first electrode, the second electrode, the perovskite sensing layer, and the functional layer described above, a secondary configuration for detecting gas and a secondary configuration for detecting radiation used in the art. Specifically, the configuration for detecting gas used in the art may be operatively connected to the first electrode, and the configuration for detecting radiation used in the art may be operatively connected to the second electrode. Thereby, the material for a gas and radiation detection sensor can present a gas sensing result detected in the first region and a radiation sensing result detected in the second region to a user.

According to an embodiment of the present disclosure, the material for a gas and radiation detection sensor may sense gas in the first region of the perovskite sensing layer provided with the functional layer, and may sense radiation in the second region of the perovskite sensing layer in which the functional layer is not provided. Thereby, there is an advantage in that a sensing effect for each of the gas and the radiation can be maximized by preventing sensing degradation due to interference between the gas and the radiation.

An embodiment of the present disclosure provides a gas and radiation detection sensor including the material for a gas and radiation detection sensor.

The gas and radiation detection sensor includes the material for a gas and radiation detection sensor described above, thereby being capable of effectively detecting gas and radiation. In addition to including the material for a gas and radiation detection sensor according to the above-described embodiment, the sensor may include, without limitation, configurations of sensors used in the art.

Hereinafter, the present disclosure will be described in more detail by way of examples. These Examples are merely for illustrative purposes and are not intended to limit the present disclosure.

### Example: Material for Gas and Radiation Detection Sensor

A material for a gas and radiation detection sensor having a structure shown in FIGS. 2 and 3 was manufactured by the following method.

### Manufacturing of Perovskite Sensing Layer

Methylammonium bromide (MABr) and lead bromide (PbBr₂) were prepared as precursor materials. Thereafter, a solute in which methylammonium bromide and lead bromide were mixed at a molar ratio of 1:1 was dissolved in an N, N-dimethylformamide (DMF) solvent at a concentration of 1.2 M to prepare a perovskite precursor solution.

Thereafter, perovskite crystals were generated by controlling a temperature of the prepared precursor solution from room temperature (about 20 °C) to 60 °C at a heating rate of 1 °C/min using an inverse temperature crystallization method. In this case, a perovskite sensing layer includes a MAPbBr₃ (MA: methylammonium ion) single crystal.

Thereby, a perovskite sensing layer having a thickness of about 5 mm was manufactured.

### Manufacturing of Functional Layer

Molybdenum trioxide (MoO₃) was prepared as a functional material. Thereafter, MoO₃ was coated on the first region of one surface of the perovskite sensing layer by a vacuum thermal deposition method (pressure: 10⁻⁶ Torr, deposition rate: 0.2 Å/s) to form a functional layer. In this case, a thickness of the functional layer was 5 nm.

### Formation of First Electrode

Silver (Ag) was coated on one surface of the functional layer by a vacuum thermal deposition method (pressure: 10⁻⁶ Torr, deposition rate: 0.2 Å/s) to manufacture a first electrode. In this time, a thickness of the first electrode was 70 nm.

### Formation of Second Electrode

In the same manner as the first electrode, silver (Ag) was coated on the second region of one surface of the perovskite sensing layer to manufacture a second electrode. In this time, a thickness of the second electrode was 70 nm.

### Comparative Example

In comparison with the above Example, a material for a gas and radiation detection sensor was manufactured in the same manner as in the above Example, except that a functional layer was not formed.

That is, the first electrode was formed in the first region of one surface of the perovskite sensing layer without providing a functional layer. The second electrode was formed in the second region of one surface of the perovskite sensing layer.

### Experimental Example

With respect to the materials for a gas and radiation detection sensor manufactured in the above Example and Comparative Example, a gas sensing performance evaluation and a radiation sensing performance evaluation were conducted by the following methods.

### Gas Sensing Performance Evaluation

Using a mass flow controller (MFC), a change in a current signal according to gas exposure was checked under a condition of NO₂ gas at 15 ppm in a state where 1 V was applied utilizing a potentiostat (manufacturer: BioLogic; model name: VSP) equipment.

FIG. 4 is a diagram illustrating results of the gas detection performance evaluation of the material for a gas and radiation detection sensor manufactured in the Example of the present disclosure.

Referring to FIG. 4, it was confirmed that, when the material for a gas and radiation detection sensor manufactured in the Example was exposed to NO₂ gas and when the exposure was stopped, a current signal was clearly changed.

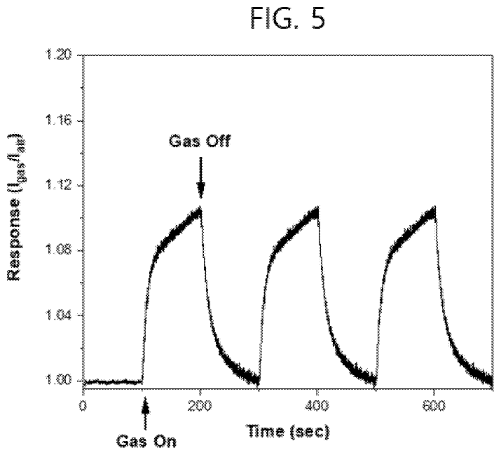

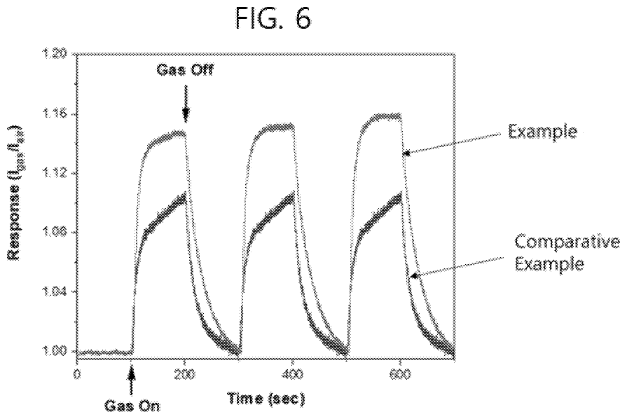

FIG. 5 is a diagram illustrating results of the gas detection performance evaluation of the material for a gas and radiation detection sensor manufactured in the Comparative Example, and FIG. 6 is a diagram comparing results of the gas detection performance evaluation of the material for a gas and radiation detection sensor manufactured in the Example of the present disclosure and the material for a gas and radiation detection sensor manufactured in the Comparative Example.

Referring to FIGS. 5 and 6, it was confirmed that, when the material for a gas and radiation detection sensor manufactured in the Comparative Example was exposed to NO₂ gas and when the exposure was stopped, there was a change in a current signal, but a width of the change was small compared to the Example.

Accordingly, it can be seen that the material for a gas and radiation detection sensor according to the Example in which the functional layer is provided has more excellent gas sensing capability compared to the Comparative Example.

### Radiation Sensing Performance Evaluation

Under a gamma-ray irradiation condition, a change in a current signal according to gamma-ray exposure was checked in a state where 10 V was applied utilizing a potentiostat (manufacturer: BioLogic; model name: VSP) equipment.

In this case, a radiation source was set to Gamma-ray 662 keV and 2.39 mSv/h.

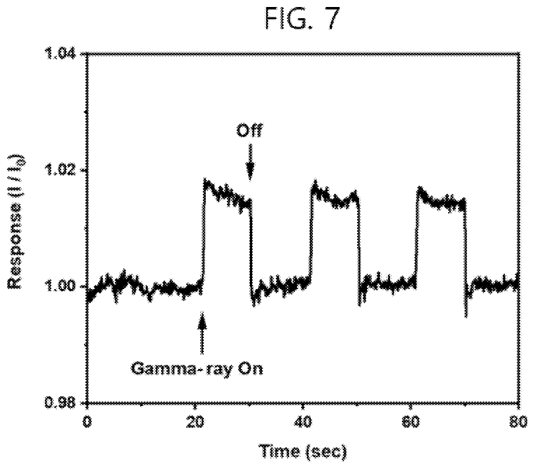

FIG. 7 is a diagram illustrating results of the radiation detection performance evaluation of the material for a gas and radiation detection sensor manufactured in the Example of the present disclosure.

Referring to FIG. 7, it was confirmed that, when the material for a gas and radiation detection sensor was exposed to gamma rays and when the exposure was stopped, a current signal was clearly changed.

Accordingly, it can be seen that the material for a gas and radiation detection sensor and the sensor including the same according to an embodiment of the present disclosure can effectively detect gas and radiation.

### DESCRIPTION OF REFERENCE NUMERALS

1: Material for gas and radiation detection sensor
110: First electrode
111: 1-1 unit electrode 112: 1-2 unit electrode
120: Second electrode
121: 2-1 unit electrode 122: 2-2 unit electrode
200: Perovskite sensing layer
300: Functional layer

What is claimed is:

1. A material for a gas and radiation detection sensor comprising:
a first electrode;
a second electrode;
a perovskite sensing layer including perovskite and configured to sense gas and radiation; and
a functional layer provided on one surface of the perovskite sensing layer and including a functional material.

2. The material of claim 1, wherein the perovskite sensing layer comprises at least one of perovskites represented by Chemical Formula 1 to Chemical Formula 4 below:
ABX₃ [Chemical Formula 1]
A′₂Aₙ₋₁BₙX₃ₙ₊₁ [Chemical Formula 2-1]
A″Aₙ₋₁BₙX₃ₙ₊₁ [Chemical Formula 2-2]
A₄BX₆ [Chemical Formula 3-1]
AB₂X₅ [Chemical Formula 3-2]
A₃B₂X₉ [Chemical Formula 4]
wherein:
A, A′, and A″ each independently comprise at least one of an organic ammonium ion and an alkali metal,
B comprises at least one of Pb, Sn, Bi, and Sb,
X is a halogen anion, and
n is 0 or an integer of 1 to 10.

3. The material of claim 1, wherein the functional layer is provided on a part of one surface of the perovskite sensing layer.

4. The material of claim 1, wherein the functional material comprises an ammonium halide-based compound.

5. The material of claim 1, wherein the functional material comprises a gas sensing-enhancing material including at least one of a thiol group and a carboxyl group.

6. The material of claim 1, wherein the functional material comprises at least one of an electron transport-enhancing material and a hole transport-enhancing material.

7. The material of claim 1, wherein the functional material comprises a radiation-resistant material including a lanthanide metal ion.

8. The material of claim 1, wherein the functional layer has a thickness of 1 nm or more and 50 nm or less.

9. The material of claim 1, wherein the first electrode comprises a 1-1 unit electrode and a 1-2 unit electrode, and the second electrode comprises a 2-1 unit electrode and a 2-2 unit electrode, and
wherein the perovskite sensing layer and the functional layer are provided between the 1-1 unit electrode and the 1-2 unit electrode, and the perovskite sensing layer is provided between the 2-1 unit electrode and the 2-2 unit electrode.

10. The material of claim 1, wherein the first electrode and the second electrode are provided to face each other,
the first electrode is provided on one surface of the functional layer provided on a first region of the perovskite sensing layer, and the second electrode is provided on a second region of the perovskite sensing layer.

11. A gas and radiation detection sensor comprising the material according to claim 1.
